(19) United States
(12) Patent Application Publication
(10) Pub. No.: US 2005/0032876 A1
(43) Pub. Date: Feb. 10, 2005

(54) Pharmaceutical compositions comprising sumatriptan and metoclopramide, and methods of use thereof

(76) Inventor: Guy Vergnault, Loechle (FR)

Correspondence Address: Daniel F. Coughlin, Esq.;Mintz Levin Cohn Ferris Glovsky and Popeo PC, Chrysler Center, 666 Third Avenue, New York, NY, 10017, US

(21) Appl. No.: 10/888,324

(22) Filed: Jul. 9, 2004

(30) Foreign Application Priority Data
Jul. 31, 2003 (GB) 0317904.1

(51) Int. Cl. A61K 9/20; A61K 31/405

(52) U.S. Cl. 514/419; 424/464

(57) ABSTRACT

The invention is concerned with a unit dosage form containing sumatriptan and metoclopramide. The unit dosage form is so configured that a portion of the sumatriptan dose is provided in a first phase adapted for immediate release of sumatriptan, and a second portion is contained in a second phase adapted for the sustained release of sumatriptan.

This application claims priority to UK Application No. 0317904.1, filed Jul. 31, 2003, the contents of which are incorporated by reference herein.

### FIELD OF THE INVENTION

This invention is concerned with improvements in the treatment of migraine by administering to a patient in need thereof, a newly disclosed unit dosage form containing sumatriptan and metoclopramide.

### Background

Sumatriptan is a selective 5HT₁, receptor agonist. It has been marketed for many years as a safe and effective treatment for migraine under the trade mark IMITREX. However, sumatriptan is rapidly pre-systemically metabolized and as such it has a very short half-life of the order of only about 2 hours. Accordingly, IMITREX, which is provided as an immediate release solid oral dosage form, is recommended to be initially dosed at 25 mg and thereafter at 2 hour intervals up to a maximum 300 mg per 24 hour period.

Such multiple daily dosing is certainly inconvenient for a patient, and can result in missed doses or made-up doses due to patient non-compliance with the recommended regimen. When sequential immediate release doses are taken in a timely manner, there are peaks and troughs in blood plasma levels consistent with the taking of each dose. However, when not administered on schedule, the resulting peaks and troughs can result in toxic doses or sub-therapeutic doses with no patient benefit. Accordingly, there is a need for improved medicaments containing sumatriptan.

It would seem that a sustained release approach to the formulation of sumatriptan would be indicated to achieve therapeutic plasma levels in patients over an extended period of time in order to obviate the need of frequent, multiple dosing. However, it is well known that drugs with very short half-lives (i.e. about 2 hours or less) are poorly suited to this formulation approach. Still further, a sustained-release approach is also generally unsuitable for drugs that are absorbed at varying rates in the different regions of the gastrointestinal tract. In this regard, studies of the gastrointestinal absorption characteristics of sumatriptan show that its extent of absorption varies with regional differences in the gastrointestinal tract. Further still, patients require rapid relief from the debilitating symptoms of migraine, and a sustained release formulation is not optimal in providing this. For these reasons, it is submitted that a skilled person would dismiss as impracticable the use of a sustained release formulation for sumatriptan.

European patent application EP-A-1126840 discloses a combination of sumatriptan with metoclopramide. It is stated that such a formulation can increase blood concentrations of 5HT₁ receptor agonists. Furthermore, metoclopramide has the added benefit in that it is an anti-emetic. However, while this reference mentions increased blood concentrations of 5HT₁, receptor agonists, it does not mention how one can prolong the duration of therapeutic blood levels with a unit dosage form, and it is silent as to the advantages in terms of prolonged therapeutic blood level concentrations that can be achieved by judicious selection of formulation principles and excipients. In fact, this patent application is quite undiscriminating in this regard, suggesting that the combination may be formulated in any number of ways, apparently with the same attendant advantages. Thus, it is stated that the combination can be administered orally, bucally, intranasally, parenterally (e.g., intravenously, intramuscularly or subcutaneously), rectally, or in a form suitable for inhalation or insufflation. In so far as the citation does refer to oral dosage forms, it refers to tablets or capsules that are prepared using conventional techniques and contain conventional excipients, i.e. binders, fillers, disintegrants and wetting agents that are commonly employed in conventional immediate release dosage forms. In particular, there is no suggestion of using excipients that control the release rate of active substances.

### DESCRIPTION OF THE INVENTION

The present invention is predicated on the surprising effects that can be achieved when using metoclopramide in combination with sumatriptan, as well as formulating sumatriptan in a sustained release environment.

It is surprising that by combining sumatriptan with another pharmaceutically active compound—metoclopramide—applicant is able to provide a formulation based on the principle of sustained release that delivered therapeutic levels of sumatriptan over an extended period of time compared with conventional immediate release formulations. Furthermore, by including a conventional immediate release component to the formulation, applicant is able to additionally provide a medicament that is capable of providing fast relief of symptoms of migraine sufferers.

Metoclopramide is a benzamide derivative that is a dopamine antagonist and 5HT₄ agonist. It is an effective antagonist of emesis. Its properties are well reported in the literature and require no further discussion herein.

The invention, in part, exploits metoclopramide's effect on the motility of the muscles of the gastrointestinal tract to manipulate the transit time of a dosage form passing through the gastrointestinal tract. Metoclopramide can be used to effectively influence the residence time of the dosage form in a region of the gastrointestinal tract most amenable to absorption of sumatriptan, and where the rate of pre-systemic metabolism is relatively low. In this region, it is therefore possible to use sustained release formulation principles to deliver sumatriptan over prolonged periods of time, to provide the attendant advantages described above.

These and other benefits are provided according to the invention that in a first aspect provides a unit dosage form containing sumatriptan and metoclopramide, the unit dosage form comprising a first phase containing sumatriptan and being adapted for immediate release of sumatriptan, and a second phase containing sumatriptan and being adapted for sustained release of the sumatriptan.

Unit dosage forms of the present invention quickly achieve therapeutic blood levels to provide rapid relief of symptoms through the release of sumatriptan from the immediate release phase upon contact with biological fluids in the stomach, and sustains these levels over a longer period of time than heretofore achieved without repeated dosing by virtue of the release of sumatriptan from the sustained release phase of the dosage form. As migraine is often associated with emesis and patients do not want to ingest anything, including pills, for fear of feeling nauseous, the present invention is advantageous because it enables the patient to take dosages at more spaced-apart intervals without a break-through of symptoms.

Metoclopramide may be added to the first and/or second phase. However, given the desire to arrest the symptoms of emesis as quickly as possible, and given that the effect on the motility of the muscles in the GI tract appear to be insensitive to the rate of release, it is preferred to add the metoclopramide to the first phase only.

“Immediate release” is an art recognized term. A formulation is considered to act as an immediate release formulation if it meets disintegration and/or dissolution requirements for immediate release solid oral dosage forms as set forth in the United States Pharmacopeia. Preferably, the immediate release phase of the dosage forms of the present invention disintegrates within about 15 minutes in an aqueous medium. Disintegration does not imply complete dissolution of the drug contained in the phase. Complete disintegration is a state in which any residue of the phase remaining in the test apparatus is a soft mass having no palpably firm core, as is more fully described in the Pharmacopeia.

An apparatus for determining disintegration rates is defined in USP 26/NF 21 under chapter 701. Therein an apparatus is described that consists of a basket-rack assembly, a 1000 ml beaker having an inside volume for receiving the immersion fluid, a thermostatic arrangement for heating said fluid between 35 and 39 degrees; and a device for lowering the basket into the immersion fluid at a constant frequency of between 29 and 32 cycles per minute. The apparatus is more fully described in the USP monograph aforementioned (which is hereby incorporated by reference) and needs no further elaboration here. Likewise, a procedure for carrying out the disintegration step is disclosed in the aforementioned monograph which is incorporated herein by reference.

The dissolution characteristics of the first (immediate release) phase are preferably such that the phase displays 75% dissolution of sumatriptan within 30 minutes in a buffered solution at a temperature of 37° C. with a paddle rate of 100 rpm using the paddle method (apparatus No. 2). USP26/NF21 (711 “Dissolution”) describes compendial test methods and apparatus, which enable investigators to assess that dissolution requirements are met, and this document is incorporated herein by reference.

The term “sustained release” used in relation to the second phase means that the phase is adapted to release drug within a certain time, or certain location to accomplish a therapeutic objective not possible using conventional immediate release formulations.

The first phase (immediate release phase) contains sumatriptan in an amount adapted to provide rapid relief of migraine symptoms. Preferably it is employed in an amount of 25 mg to 150 mg, more particularly 25 to 100 mg per unit dosage form. Preferably the first phase contains 25 to 75% of the total sumatriptan loading in the unit dosage form. The first phase preferably may additionally contain metoclopramide. When metoclopramide is employed, it may be present in amounts of 5 to 30 mg per unit dosage form, more preferably 10 to 20 mg per unit dosage form. Preferably the first phase contains from 50 to 100% of the total metoclopramide in the unit dosage form.

Other tablet excipients may be employed in the first phase, which are commonly employed in dosage forms for immediate release of active agents, including diluents or fillers, which add bulk to a formulation to enable formulations of a desired size to be prepared; binders or adhesives, which promote the adhesion of the particles of a formulation to maintain the integrity of the dosage form; disintegrants or disintegrating agents, which promote the break-up of the dosage form after ingestion to make the actives more readily available; anti-adherents, glidants or lubricants, which enhance the flow of the tableting materials, for example into tablet dies, prevent sticking of the formulation to tablet-making machinery; and miscellaneous adjuvants such as colorants and flavorants.

Suitable diluents include pharmaceutically acceptable inert fillers such as microcrystalline cellulose, lactose, dibasic calcium phosphate, saccharides, and/or mixtures of any of the foregoing. Examples of diluents include microcrystalline cellulose such as Avicel ph 112, Avicel pH101 and Avicel pH 102; lactose such as lactose monohydrate, lactose anhydrous, and Pharmatose DCL 21; dibasic calcium phosphate such as Emcompress; mannitol; starch; sorbitol; fructose; sucrose; and glucose. Diluents are carefully selected to match the specific formulation with attention paid to the compression properties. The diluent is preferably used in an amount of 10% to 90% by weight, more particularly 50% by weight, of the immediate release phase.

Suitable lubricants and glidants, including agents that act on the flowability of the powder to be compressed are, for example, colloidal silicon dioxide such as Aerosil 200; talc; stearic acid, magnesium stearate, calcium stearate, sodium stearyl fumarate, polyethylene glycol and sodium lauryl sulphate. The lubricant is preferably used in an amount of 0.5 to 2% by weight, in particular 1% by weight, of the immediate release phase.

Suitable binders include polyethylene glycols such as PEG 6000; cetostearyl alcohol; cetyl alcohol; polyoxyethylene alkyl ethers; polyoxyethylene castor oil derivatives; polyoxyethylene sorbitan fatty acid esters; polyoxyethylene stearates; poloxamers; waxes, alginic acids and salts thereof; HPC; HPMC; methylcellulose; maltodextrin and dextrin; povidone; gums; starch and modified starches. The binder may be used in an amount of 2 to 10% by weight, more particularly 5% by weight, of the immediate release phase.

Suitable disintegrants include sodium starch glycolate, such as EXPLOTAB® crospovidone such as Kollidon CL, polyplasdone XL, sodium carboxymethylcellulose, sodium croscarmellose such as AcDiSol, and starch. The disintegrant may be used in an amount of 2 to 10% by weight, more particularly 5% by weight, of the immediate release phase.

The second phase (sustained release phase) contains sumatriptan in an amount of 25 to 100 mg. more particularly 25 to 75 mg, per unit dosage form. Preferably, the second phase contains 25 to 75% of the total sumatriptan loading in the unit dosage form. If metoclopramide is employed in the second phase, preferably it represents up to from 0 to 50% of the total loading of metoclopramide in the unit dosage form. The sustained release phase may additionally contain any of the aforementioned adjuvants referred to in relation to the immediate release phase in the amounts mentioned.

Additionally, the sustained release phase contains a release-rate-controlling agent. This rate-controlling agent may be provided in the aforementioned matrix in which the sumatriptan is embedded. Alternatively (or additionally), the rate-controlling agent may be in the form of a coating that surrounds a conventional immediate release formulation substantially as herein above described.

The term “release-rate controlling agent” includes any agent that controls the rate of release of an active substance in terms of duration or location in order to give a therapeutic effect not possible with a conventional immediate release formulation, and includes hydrophilic polymers, hydrophobic polymers or mixtures thereof, or copolymers thereof, or mixtures of these polymers and copolymers.

Examples of release-rate controlling agents to be used in this invention include hydroxyalkylcellulose, such as hydroxypropylcellulose and hydroxypropylmethylcellulose; poly(ethylene)oxide; alkylcellulose such as ethycellulose and methylcellulose; carboxymethylcellulose; hydrophilic cellulose derivatives; polyethylene glycol; polyvinylpyrrolidone; cellulose acetate; cellulose acetate butyrate; cellulose acetate phthalate; cellulose acetate trimellitate; polyvinylacetate phthalate; hydroxypropylmethylcellulose phthalate; hydroxypropylmethylcellulose acetate succinate; poly(alkyl methacrylate); and poly (vinyl acetate). Other suitable hydrophobic polymers include polymers or copolymers derived from acrylic or methacrylic acid esters, copolymers of acrylic and methacrylic acid esters, zein, waxes, shellac and hydrogenated vegetable oils.

When provided in or as a matrix, the release-rate-controlling agent preferably includes a hydroxypropyl methylcellulose (HPMC), a hydroxypropyl cellulose (HPC), a poly(ethylene oxide), an ethylcellulose or a combination thereof present in an amount of 10 to 90% based on the weight of the sustained release phase.

Preferred types of HPMC for use in accordance with the invention are those sold under the trademark METHOCEL (Dow Chemical Co.). Suitable METHOCELs include the K grades such as METHOCEL K15M, METHOCEL K100M, METHOCEL K100LV and METHOCEL K4M. Other suitable METHOCELS include the E, F and J grades.

As HPCs there can be those sold under the trademark KLUCEL (Hercules, Inc.) or equivalents. Suitable KLUCELS include KLUCEL LF, KLUCEL JF, KLUCEL GF, KLUCEL MF and KLUCEL HF.

As poly(ethylene oxide)s there may be mentioned those sold under the trademark SENTRY POLYOX (Union Carbide Corp.) or equivalents. Suitable POLYOXS include the POLYOX WSR grades such as POLYOX WSR Coagulant, POLYOX WSR-301, POLYOX WSR-303, POLYOX WSR N-12K, POLYOX WSR N-60K, POLYOX WSR-1105, POLYOX WSR-205 and POLYOX WSR N-3000.

As ethylcelluloses for use in accordance with the invention there can be mentioned those sold under the trademark ETHOCEL (Dow Chemical Co.) or equivalents.

The hydroxypropylmethylcelluloses preferably have a viscosity (2 wt % solution at 20.degree. C.) of about 5 to 100,000 cps, preferably 4,000 to 100,000 cps. Especially suitable are METHOCEL K types or their equivalents. The hydroxypropylcelluloses used according to the invention preferably have a number average molecular weight of about 80,000 to 1,150,000, more preferably 80,000 to 600,000. Especially suitable is KLUCEL LF, which has a molecular weight of 100,000.

Poly(ethylene oxides) preferably have number average molecular weights of about 100,000 to 7,000,000, more preferably 900,000 to 7,000,000. Especially suitable is POLYOX WSR Coagulant, which has a molecular weight of 5,000,000. The ethylcelluloses used according to the invention preferably have a viscosity of about 3 to 110 cps, more preferably 7 to 100 cps.

The release kinetics of the sustained release phase vary according to the amount of release-rate-controlling polymer that is employed in the sustained release phase. Preferably, one employs 10 to 60 wt % of the sustained release phase.

A suitable sustained release phase may be formed from the following:
(a) Hydrophilic or hydrophobic polymers, such as gums, cellulose ethers, acrylic resins and protein derived materials. Of these polymers, the cellulose ethers, especially hydroxyalkylcelluloses and carboxyalkylcelluloses, are preferred.
(b) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. Hydrocarbons having melting points of between 25.° C. and 120.° C. are preferred. The sustained release phase may contain up to 50% (by weight) of at least one digestible, long chain hydrocarbon.
(c) Polyalkylene glycols. The sustained release phase may contain up to 25% (by weight) of at least one polyalkylene glycol.

A particularly suitable sustained release phase may comprise at least one water soluble hydroxyalkyl cellulose, at least one C₁₂-C₃₆, preferably C₁₄-C₂₂, aliphatic alcohol or glyceryl ester, e.g. tribehenin and, optionally, at least one polyalkylene glycol. The at least one hydroxyalkyl cellulose is preferably hydroxypropyl methylcellulose as described herein above. The at least one aliphatic alcohol may be, for example, lauryl alcohol, myristyl alcohol or stearyl alcohol. The at least one polyalkylene glycol may be, for example, polypropylene glycol or, which is preferred, polyethylene glycol. The number average molecular weight of the at least one polyalkylene glycol is preferably between 1,000 and 15,000 especially between 1,500 and 12,000.

When the sustained release phase is to include a coating of sustained release agent, that agent may be selected from any suitable release-rate-controlling agent including methyl cellulose, ethyl cellulose, hydroxypropyl cellulose, hydroxypropylmethyl cellulose, hydroxybutylmethyl cellulose, cellulose acetate, cellulose propionate (lower, medium or higher molecular weight), cellulose acetate propionate, cellulose acetate butyrate, cellulose acetate phthalate, carboxymethyl cellulose, cellulose triacetate, cellulose sulphate sodium salt, polymethylmethacrylate, polyethylmethacrylate, polybutymethacrylate, polybutymethacrylate, polyisobutymethacrylate, polyhexomethacrylate, polyisodecylmethacrylate, poly(lauryl methacrylate), poly(phenyl methacrylate), polymethalacrylate, polyisopropylacrylate, polyisbutalacrylate, polyoctadcylacrylate, polyethylene (low or high density), polypropolyne, polyethylene glycol, polyethylene oxide, polyethylene terephthalate, polyvinyl alcohol, polyvinyl isobutyl ether, polyvinyl acetate, polyvinyl chloride and polyvinyl pyrrolidone. Examples of suitable copolymers include: butylmethacrylate/isobutylmethacrylate copolymer, high molecular weight, methylvinyl ether/maleic acid copolymer, methlvinyl ether/maleic acid, monoethyl ester copolymer, methylvinyl ether/malec and anhydride copolymer and vinyl alcohol/vinyl acetate copolymer. Examples of suitable biodegradable polymers include: polylactides, polyglycolides, polyethylene terathatic and polyeurathine. Examples of suitable acrylate and methacrylate are polyacrylic and methacrylic polymer such as those sold under the trademark EUDRAGIT®.

A typical sustained release coating may include:—
a) wax, either alone or in admixture with a fatty alcohol,
b) shellac or zein,
c) a water insoluble cellulose, especially ethyl cellulose, and
d) a polymethacrylate, especially EUDRAGIT®.

Preferably, the film coat comprises a mixture of the water insoluble material and a water soluble material as a channelling agent. The ratio of water insoluble to water soluble material is determined by, amongst other factors, the release rate required and the solubility characteristics of the materials selected. The water soluble material may be, for example, polyvinylpyrrolidone or, which is preferred, a water soluble cellulose, especially hydroxypropylmethyl cellulose, or a soluble, non-polymeric material such as lactose. Suitable combinations of water insoluble and water soluble materials for the film coat include shellac and polyvinylpyrrolidone or, which is preferred, ethyl cellulose and hydroxypropylmethyl cellulose. When a sustained release coating is employed, preferably it comprises from about 5 to 30% by weight of the sustained release phase.

The unit dosage forms of the present invention may be presented in the form of a compressed tablet consisting of adjacent layers of immediate release and sustained release phases. Alternatively, they may be presented as granules, spheroids or pellets, a population of which are adapted for sustained release, and a population adapted for immediate release. Such granules, spheroids or pellets may be provided loosely in a capsule form, or they may be compressed into a tablet.

When the unit dosage form is presented as a compressed tablet consisting of adjacent layers of immediate release and sustained release phases, these layers may be presented as adjacent layers arranged laterally, or concentrically. Each unit dosage form may contain two or more adjacent layers. When arranged concentrically, the unit dosage form preferably consists of a central core consisting of the sustained release phase, and surrounding that central core, a first phase adapted for immediate release. When the unit dosage form is presented as a plurality of granules, spheroids or pellets, they may be formed of matrix materials as aforementioned, or they may be formed of inert non-pareils, coated or embedded with respectively the first and second phases.

The skilled person will appreciate that many physical embodiments are possible and the only limitation to the formulation is that it must contain the two phases aforementioned.

Unit dosage forms, and/or the phases of the unit dosage forms of the present invention. may be coated with coating materials to achieve all manner of desired effects. For examples coatings may be provided to achieve an aesthetic effect (e.g., an attractive color or pleasant taste) or information effect (e.g., a coating may be colored to act as a visual cue for a patient identification of the correct medicament. Coatings may also be over-written with information relating to the dosage); or a functional effect such as a handling effect (e.g., a smooth coating for ease of swallowing), or a stability effect (e.g., a moisture or light barrier during storage).

In order to facilitate the preparation of a unit dosage form according to this invention there is provided, in a further aspect of the present invention, a process for the preparation of a unit dosage form according to the present invention.

When presented in the form of a compressed tablet comprising adjacent layers of immediate release and sustained release phases, the unit dosage forms may be made by compression methods by the application of high pressures to powders or granulates utilizing steel punches and dies. In this manner a wide variety of shapes, sizes and surface markings can be formed depending on the size and design of the punches and dies employed. On an industrial scale they may be produced using rotary presses suitable for producing multilayer tablets, e.g., a Layer-Press, Manesty, Liverpool, United Kingdom. Presses generally operate at pressures of about 1000 to about 5000 kg/cm².

Compressed tablets may be made by three methods well known in the art: wet granulation; dry granulation; and direct compression.

If powders are used that are free-flowing and cohesive, compression can take place using direct compaction in a tableting machine according to methods known in the art. More usually however, tablets are formed by compression of granulates. Granulates may be formed according to dry granulation or wet granulation methods.

Dry granulation (formed by roller compaction or slugging) involves the compaction of powders at high pressure into large tablet compacts. Granulates may also be formed by extruding powders between rollers to form thin ribbons. These compacts are then milled and screened to form granulates of the desired particle size.

Wet granulation is a technique widely employed in the art and comprises the steps of i) weighing and blending the ingredients; ii) preparing a damp mass from the ingredients; iii) screening the mass into pellets or granules; iv) drying the granulate; v) sizing the granulate by screening; vi) adding lubricant as appropriate and blending; and vii) tableting by compression.

When presented as granules, spheroids or pellets, unit dosage forms may be prepared by forming granules according to any of the granulation techniques described above, and then optionally shaping the granules by extrusion or compression, or spheronising the granules to form pellets or spheroids. Thereafter, the granules, pellets or spheroids may be placed in capsule shell, or they may be compressed into a tablet form. Should coating of the phases or unit dosage forms be required, this can be achieved using conventional coating techniques such as spray coating, pan coating, or air suspension coating techniques.

All of the techniques discussed above are described in detail in Ansel's Pharmacetical Dosage Forms and Drug Delivery Systems, Chapter 7, Seventh Edition, 1999 (Lippincott Williams & Wilkins), which is herein incorporated by reference.

Unit dosage forms of the present invention are useful medicaments in the treatment of migraine such as acute migraine attacks with or without aura. The dosage forms are useful to retard, reverse, GF alleviate. or prevent the symptoms of migraine. Furthermore, by virtue of the use of metoclopramide, emesis, often associated with migraine attacks may be alleviated or prevented.

The appropriate dosage and the intervals between each dosage will depend upon the subject being treated and the type and severity of the condition for which the subject is being treated. Generally speaking. however, the subject will receive orally between 25 and 100 mg sumatriptan per day per 70 kg body weight, whereas metoclopramide will be administered in amounts of about 20 to 80 mg per day per 70 kg body weight.

In a particularly preferred embodiment of the present invention, a unit dosage form will contain 100 mg sumatriptan and 10 to 20 mg metoclopramide.

Upon ingestion, unit dosage forms of the present invention release a first pulse of sumatriptan to reach a therapeutic plasma concentration within a short period of time, to effect rapid pain relief. Thereafter. a second pulse reaches and maintains therapeutic plasma concentrations over a prolonged time period, thus extending the duration of pain relief compared to conventional immediate release forms of sumatriptan.

The invention has been described with reference to sumatriptan. However, the invention also applies to salts of sumatriptan, and in particular to its succinate salt. Further, the invention applies to metoclopramide as well as its salts, and in particular its hydrochloride salt form.

### EXAMPLES

The invention will now be further illustrated with reference to the following Example.

### Example 1

Tablets containing a sustained release layer containing sumatriptan succinate, and an immediate release layer containing sumatriptan succinate and metoclopramide (as the hydrochloride monohydrate), are formed according to the following procedure:

A granular mass for the sustained release layer is prepared as follows:

Preparation of a granular mass for the sustained release layer

| | |
|---|---|
| Sumatriptan Succinate | 70 parts |
| Lactose (meggle) | 40 parts |
| HPMC (METHOCEL K4M) | 40 parts |
| PVP (PLASDONE K29-32) | 6 parts |
| Magnesium Stearate (C. Erba) | 1 parts |
| Colloidal Silica (SYLOID 244 Grace GmbH) | 1 parts |

The sumatriptan, lactose, PVP and HPMC are mixed together in a high shear blender and wetted with a granulation liquid of purified water. The mixture is sieved through a 25 mesh and dried in a fluidized bed dryer to a pre-defined residual moisture level, and sieved again through the same screen before adding the magnesium stearate and silica. Thereafter, the mixture is mixed in a tumble mixer for 10 minutes. The granular mass obtained (A) exhibits good flow and compacting properties.

A granular mass for the immediate release layer is prepared as follows:

Preparation of granular mass for the immediate release layer

| | | |
|---|---|---|
| Sumatriptan Succinate | 50 | parts |
| Metoclopramide (HCl.H₂O) | 11.8 | parts |
| Lactose | 50 | parts |
| Polyvinylpyrrolidone (PLASDONE K29-32) | 8 | parts |
| Crosslinked polyvinylpyrrolidone (POLYPLASDONE XL) | 10 | parts |
| Magnesium stearate | 1.2 | parts |
| Colloidal Silica (SYLOID 244) | 1 | parts |

Sumatriptan, metoclopramide, lactose and PVP are blended together in a high shear mixer and then wetted with a purified water as a granulation liquid. The mixture is sieved through a 25 mesh screen, dried in a fluidized bed dryer to a pre-defined residual moisture content, and then sieved again through the same screen. The resultant granules are mixed with the crosslinked PVP, magnesium stearate and colloidal silica in a diffusion-type blender for 15 minutes. The granular mixture obtained (B) is free-flowing and has good compaction properties.

A Manesty Dry Cota rotary press is used for the manufacture of compressed tablets. As is well known to those skilled in the art, the rotary press consists of several feeding stations to receive the granular layer blends. The press is equipped with circular concave punches. A first hopper is filled with the mixture A, and a second hopper is filled with the mixture B. The first hopper is set to deliver 158 mg, and the second hopper set to deliver 152 mg. The operating pressure is 1500 kg/cm². The compressed tablets formed have a weight of 310 mg and contain 100 mg sumatriptan and 50 mg metoclopramide.

The release characteristics of the sumatriptan are evaluated by USP dissolution apparatus no. 2 operated at 100 rpm, and in a dissolution medium of 1000 ml water at 37 degrees centigrade.

1-9. (Canceled)

10. A unit dosage form comprising sumatriptan, or a pharmaceutically-acceptable salt thereof, and metoclopramide or a pharmaceutically-acceptable salt thereof, said unit dosage form comprising a first phase containing sumatriptan, or a pharmaceutically-acceptable salt thereof, and being adapted for immediate release of sumatriptan, or a pharmaceutically-acceptable salt thereof, and a second phase containing sumatriptan, or a pharmaceutically-acceptable salt thereof, and being adapted for sustained release of sumatriptan, or a pharmaceutically-acceptable salt thereof.

11. The unit dosage form according to claim 10, wherein the pharmaceutically-acceptable salt of sumatriptan comprises sumatriptan succinate.

12. The unit dosage form according to claim 10, wherein the pharmaceutically-acceptable salt of metoclopramide comprises metoclopramide hydrochloride or hydrochloride monohydrate.

13. The unit dosage form according to claim 10, wherein metoclopramide, or a pharmaceutically-acceptable salt thereof, comprises the first phase.

14. The unit dosage form according to claim 10, wherein metoclopramide or a pharmaceutically-acceptable salt thereof, comprises the second phase.

15. The unit dosage form according to claim 10, wherein sumatriptan is present in the first phase in an amount of about 25 mg to about 150 mg per unit dosage form.

16. The unit dosage for according to claim 10, wherein metoclopramide is present in the first phase an amount of about 5 mg to about 30 mg per unit dosage form.

17. The unit dosage form according to claim 10, wherein sumatriptan is present in the second phase in an amount of about 25 mg to about 100 mg per unit dosage form.

18. The unit dosage form according to claim 10, comprising a tablet containing the first and second phases arranged in adjacent layers.

19. A method of treating migraine, comprising the step of providing to a patient in need thereof the unit dosage form according to claim 10.

20. The method according to claim 19, wherein the unit dosage form comprises a package containing instructions for use.

21. The method according to claim 19, wherein the unit dosage form is provided orally.

22. The method according to claim 21, wherein the unit dosage form provided orally comprises sumatriptan in an amount of between about 25 and 100 mg per day per 70 kg body weight, and metoclopramide in an amount of between about 20 to 80 mg per day per 70 kg body weight.

23. A sustained-released, unit dosage formulation comprising sumatriptan, or a pharmaceutically-acceptable salt thereof, and metoclopramide or a pharmaceutically-acceptable salt thereof, said formulation comprising (i) a first phase containing sumatriptan, or a pharmaceutically-acceptable salt thereof, said first phase being adapted for immediate release of sumatriptan, or a pharmaceutically-acceptable salt thereof, and (ii) a second phase containing sumatriptan, or a pharmaceutically-acceptable salt thereof, said second phase being adapted for sustained release of sumatriptan, or a pharmaceutically-acceptable salt thereof.

24. The sustained-release, unit dosage form according to claim 23, wherein the pharmaceutically-acceptable salt of sumatriptan comprises sumatriptan succinate.

25. The sustained-release, unit dosage form according to claim 23, wherein the pharmaceutically-acceptable salt of metoclopramide comprises metoclopramide hydrochloride or hydrochloride monohydrate.

26. The sustained-release, unit dosage form according to claim 23, wherein metoclopramide, or a pharmaceutically-acceptable salt thereof, comprises the first phase.

27. The sustained-release, unit dosage form according to claim 23, wherein metoclopramide or a pharmaceutically-acceptable salt thereof, comprises the second phase.

28. A method of prolonging the delivery of sumatriptan to a patient in need thereof, comprising:
administering to the patient the unit dosage form according to claim 10 or claim 23, wherein the release of sumatriptan is sustained in the bloodstream so as to prolong the delivery of sumatriptan to the patient.
